(19) United States
(12) Patent Application Publication
(10) Pub. No.: US 2005/0118123 A1
(43) Pub. Date: Jun. 2, 2005

(54) Use of chemiluminescence in cosmetics & chromatography

(76) Inventor: Niteen Ashok Vaidya, Cupertino, CA (US)

Correspondence Address: Dr. Niteen Vaidya, 616 Stendhal Lane, Cupertino, CA, 95014, US

(21) Appl. No.: 10/981,374

(22) Filed: Nov. 3, 2004

Related U.S. Application Data
(60) Provisional application No. 60/517,040, filed on Nov. 4, 2003.

(51) Int. Cl. A61K 7/42; A61K 7/021; A61K 7/025; G01N 33/558

(52) U.S. Cl. 424/63; 424/64; 436/514

(57) ABSTRACT

Cosmetic composition consists of precursors for chemiluminescence's system is documented with novel use in hair dyes, lipsticks, sunscreen, sunblock and sunless tanning, skin brightening agents. In other application namely chromatography (experimental technique) use of chemiluminescence would make it unnecessary to use black/UV light to observe and detect chemicals in TLC (Thin layer chromatography).

### BACKGROUND

Chemiluminescence is the process in which chemical energy produced by a chemical reaction is transformed into light energy, a different form of energy. In a firefly the light-producing chemical reaction is triggered by a catalyst known as an enzyme. In a light stick, when you follow manufacturer's instructions to bend, snap, and shake the stick, you initiate chemical reaction by mixing the substances contained within separate compartments in the plastic tube. Energy generated during the course of the reaction is accepted by a dye molecule contained within the light stick and then released in the form of colored light with no accompanying heat.

The CYALUME light sticks, marketed by Omniglow Corporation, have found extensive applications beyond the initial toy and novelty market. The cool, intense light provided by a chemical light stick is ideal for emergency lighting, traffic control, and hazard identification. Numerous sports products including golf balls, footballs, hockey pucks, badminton birdies, and wiffle balls use replaceable light sticks to create innovative nighttime activities. In virtually every amusement park open past sunset, we can find varieties of glow-in-the-dark colored necklaces, bracelets, and bands. Military covert, nighttime, and emergency operations have been enhanced with the use of visible and infrared chemiluminescent products. The United States and Allied forces during Operation Desert Storm employed light sticks for underwater operations, nighttime personnel ship-to-ship identification, hazard demarcation, and color-coding of units, vehicles, and equipments. Commercial fishing fleets have found that deep-sea fish such as tuna and swordfish are attracted to the light emitted by light sticks when the fish swim in surface waters at night. In particular, the long-line fishing industry utilizes light sticks to illuminate their monofilament long lines, often extending in length up to 80 miles with more than 3000 hooks. With the variations in color, intensity, and duration of light emission possible, numerous applications may remain unexplored.

This process of “cold light” has not been used in the cosmetics field, and only a few applications in chromatography exist (such as TLC). Especially, in the field of cosmetics, these light emitting properties of various time durations are desirable.

Examples include: hair dyes, lipsticks, sunscreen, sunblock and sun-less tanning, skin brightening agents. Chemiluminescent products can be used as color cosmetics and skin treatment products, to replenish the skin's natural fluorescent glow. The present invention relates to cosmetic compositions for the application to the skin comprised of effective amounts of at least one chemiluminescent agent such as pyrogallol/esculin (natural ingredients) in combination with a cosmetically acceptable vehicle. The compositions, when applied to the skin, replenish the fluorescence that may have been lost due to chrono- or photo-aging, while not conferring any readily discernible amount of color on the skin. U.S. Pat. No. 6,313,181 B 1 procures a composition containing one fluorescent brightener to enhance skin's natural glow. Esculin can be used as an agent to brighten skin when it is mixed with peroxide and hypochlorite. The compound glows due to CL, and this in turn enhances skin highlights.

### SUMMARY OF INVENTION

From above discussion it is apparent that CL reaction is controlled (intensity and duration) by reagents, order of mixing and temperature (time duration could be anywhere from 30 seconds to as long as 8 to 10 hours). This phenomenon could be utilized to enhance and effectiveness as skin lightening agents, sunscreens and sun-less tanning systems. In skin lightening agents typically uses p-Hydroquinone or Pyrogallol. If CL could increase the brightness effect of pyrogallol for few hours that would be beneficial as skin lightening agent.

The CL reaction of Pyrogallol and peroxide could be used as hair dying system where CL reaction will add little glow for several hours.

The CL reaction of oxalate systems are well-known, If one uses oxalate system (e.g. Octyl Oxalate or Menthyl Oxalate) along with fluorescent dye to be visible by naked eyes you could effectively apply sunscreen to the desire areas without missing any spots and also oxalate system used would chemically transformed into octyl or menthyl salicylate-which are approved sunscreens would stay on the skin to act accordingly.

In sun-less tanning systems (U.S. Pat. Nos. 5,626,839 & 6,126,924) kits are provided with Black light to visualize application areas so that sun-less tanning lotion can be applied evenly.

With the present invention, black light will not be necessary since you could see the entire area of application for few minutes.

The CL reactions of Lophine (2,4,5-Triphenylimidazole) and Luminol (3-Aminophthalhydrazide) are also utilized; Lophine is used in sunless tanning lotion and Luminol in Thin layer Chromatography.

### DETAILED DESCRIPTION OF THE INVENTION

Pyrogallol was the first synthetic hair dye (occurs naturally in walnut shells which is also used in sun-less tanning lotions to give brown tinge to the skin) in the industry.

Pyrogallol was used to dye hair brown, and it was often used in combination with henna. The chemiluminescent reaction of alkaline Pyrogallol and peroxide is well known, so if peroxide is pre-mixed with Pyrogallol just prior to its application, it would bring beautiful sparkling to hair and enhance the dying process.

New advancements in the sunscreen industry bring us products that are initially bright, conspicuous colors that enable the user to more effectively protect against sunburn by allowing more complete and uniform application of the sunscreen on the skin. Sunscreens are substances or compositions applied to the skin to protect the skin from sunburn caused by the sun's ultraviolet ray. When uniformly applied to the body, sunscreens can be highly effective in protecting against sunburn. However, sunscreen failures can occur when areas of the body are missed because the sunscreen is hard to see or visualize after being applied or rubbed onto the skin. Children are at greater risk of sunburn than adults, since coverage on children's skin tends to be more incomplete, uneven or inconsistent, U.S. Pat. No. 5,747,011 teaches us sunscreen with disappearing color indicator, a water soluble dye or a blend of water-soluble dyes whose color substantially disappears when the sunscreen emulsion dries after it is spread on the skin and/or is rubbed out. However, color never totally disappears and appearance is very unappealing. WO 94/26233 teaches that phenolphthalein, a color indicator, can be added to sunscreen, provided the sunscreens are formulated at a pH greater than 9.0. However, phenolphthalein can induce skin rashes and eruptions. Additionally, sunscreens with this pH are highly basic and can be irritating to the skin. So oxalate systems or lophine could be used along with peroxide in a way that the sunscreen lotion will be visible for few minutes, just enough time to have visible effect of self-lighted lotion and will effectively prevent any areas, which might be missed by user.

WO 95/28912 teaches composite UV sunblock compositions that may contained color particles. When the sunscreen of WO 95/28912 is topically applied to the skin, the sunscreen remains visibly colored.

U.S. Pat. No. 5,626,839 teaches us sunless tanning lotion with light responsive ingredient. The composition includes a self-tanning agent together with intimately admixed fluorescent materials in a cosmetic carrier. But for this self-tanner UV or black light to see the presence and extent of lotion applied accompanies kit. So in order to avoid UV or black light altogether cosmetic composition containing chemiluminescent mixture which will lit-up for few minutes and glow will substantially disappear altogether is sought by self-tanning industry/spas/tanning salons etc. Thus sun-less tanning lotion containing oxalate/peroxide system with fluorescent dye will effectively serve the purpose of self-tanning lotion without use of any external light source.

The “Chemiluminescence” is quite extensively used in analytical chemistry such as HPLC, Capillary Electrophoresis, DNA analysis etc. but never been explored thoroughly as substitute for UV (black light) in TLC analysis. Thin layer chromatography is used extensively all over the world as quick analysis of organic reactions where silica gel is coated with fluorescent agent and organic mixture is spotted on the plate with UV-absorbing components, and when it is view under UV-light whole plate get illuminated excepts those spots where UV absorbing material is present (they appear as dark spots).

With present invention after developing a TLC plate, it can be dipped into chemiluminescent solution to visualize the dark spots without use of external UV-source. Chemiluminescence (CL) is the emission of the electromagnetic (ultraviolet, visible, or near infrared) radiation by molecules or atoms resulting from a transition from an electronically excited state to a lower state (usually the ground state) in which the excited state is produced in a chemical reaction. The CL phenomenon is relatively uncommon because, in most chemical reactions, excited molecules lose their excitation energy via nonradiactive pathways (e.g., heat). This type of luminescence provides interesting cosmetic/analytical applications.

For a reaction to produce detectable CL emissions, it must fulfill the following conditions: 1) it should be exothermic so that sufficient energy for an electronically excited state to be formed (at least 180 kJ/mol for emission in the visible region) can be provided; 2) there should be suitable reaction pathways for the excited state to be formed; and 3) a radiactive pathway (either direct or via energy transfer to a fluorophore) for the excited state to lose its excess energy should exist.

One key aspect of CL, techniques is the transient signal that results from the underlying spectroscopic, chemical, and physical kinetics. This is primarily the result of the spectroscopic phenomenon (i.e., the emission of light by a molecule in an excited electronic state on return to its ground state) being intrinsically kinetic in nature. Because excited CL states are produced by chemical reaction, chemical kinetics is also involved in the process. In fact, the intensity of the CL signal l_{CL}, is related to the reaction rate v, via the CL quantum yield, φ. The way the ingredients of aCL reaction are mixed in an aqueous medium also influences the CL signal, particularly its initial portion. As a result, the physical kinetics inherent in the fluid dynamics must also be considered.

The typical profile of a CL transient signal (a plot of CL intensity vs time) is a kinetic response curve that corresponds to a first-order sequence of two consecutive steps, namely: 1) generation of the light-emitting product by mixing of the chemical ingredients (the substrate and oxidant), and 2) formation of the end product. The rate at which each step takes place depends on the formation and decay rate constants, k₁ and k₂, which corresponds to the rising and falling portion, respectively, of the transient signal. Physical kinetics plays a roll in the prior mixing process, whereas chemical and spectroscopic kinetics is involved in both steps.

The formation of light emitter (i.e., an excited product) can be accomplished by mixing the substrate and oxidant in the presence of catalyst or cofactor.

One must distinguish between two different alternatives: direct and indirect CL. In direct CL, the excited product returns to its ground state to give the starting product and emitted light. In indirect CL, also referred to as “energy-transfer process,” the excited product interacts with a fluorophore, which must be a fluorescent molecule, to form the product; simultaneously, the fluorophore is promoted to its excited state, from which it subsequently returns to its ground state with light emission. In direct CL methods substrate and oxidant is sometimes accompanied by inhibitor that decreases the intensity of the CL signal. This has helped expand the scope of direct methodologies, which was NAV formerly restricted to the few available CL reactions. In indirect CL methods, fluorophore accepts emitted energy and perform accordingly. These methods have a broader scope than their direct counterparts as a result of the wide variety of organic substrates could be used.

The most widely used chemical systems in direct CL methods are based on classical substrates such as luminol, lophine or pyrogallol, which are oxidized with hydrogen peroxides. In indirect CL reactions, aryl oxalates are common substrates such as bis (2,4,6-trichlorophenyl)oxalate (TCPO) and bis(2,4-dinitrophenyl)oxalate (DNPO), which are oxidized with hydrogen peroxide. In this process, which is known as the peroxyoxalate-CL (PO-CL) reaction, the fluorophore ligand is a native or derivatized fluorescent organic substance such as polynuclear aromatic hydrocarbon, dansylamino acid, carboxylic acid, phenothiazine, or catecholamines, for example. The mechanism of the reaction between aryl oxalates and hydrogen peroxide is believed to generate dioxetane-1,2-dione, which may itself decomposes to yield an excited-state species. Its interaction with a suitable fluorophore results in energy transfer to the fluorophore.

For all of these systems peroxide is kept in the separate compartment then the rest of the cosmetic mixture in the dual compartments of the container. And it is mixed together just before it is applied to the general areas.

For skin lightening agents/bleach or hair dye (traditionally pyrogallol is applied first followed by hydrogen peroxide) in present invention premixed ingredients with certain concentration is applied and follow the direction for appropriate usage.

NAV

For sunscreens and sun-less tanning agents, CL properties of oxalate systems will be explored and utilized along with esculin (Horse-chest nut tree) or umbelliferone or any other equivalent as fluorescent dye to brighten the area of application. The esculin is used in the field of cosmetic as skin-protectant. This imparts fluorescent effect due to part of the structure related to esculetin (coumarin). In sun-less tanning lotion walnut extract can be used which imparts brown color to the skin due to presence of pyrogallol in the walnuts. So if walnut extract is mixed with peroxide & oxalate system in sun-less tanning lotion, along with some DHA and some fluorescent dye such as coumarin that would be effective tanning lotion which is self-lighted as well.

The oxalates, which are used in preparations, would be converted to salicylates, which are sunscreens approved by FDA.

In chromatography application such as TLC, mixture of hypochlorite and hydrogen peroxide or derivative of the same is applied/sprayed at the development step, the fluorescent dye (coumarin-1) will shine from the energy generated from the reaction of hypochlorite and peroxide and in order to visualize UV absorbing material in it (which is usually done by observing the plate under UV light). Thus visualization of Plates can be accomplished without UV light source or the chamber.

1. A method self-illuminating system for cosmetic applications such as sunscreen, sunblock and sun-less tanning system where external light source is not required to view field of application.

2. Skin brightening agents, hair dyes, lipsticks, lip gloss and other facial and skin make-up articles.

3. compounds which could be used for this application are: Lophine, Luminol, Isoluminol, Lucigenin, Acridinium esters, Pyrogallol, Resorcinol, P-dihroxybenzene, Aesculin (Esculin), Siloxene, Bis-(2,4,6-Trichlorophenyl)Oxalate, Bis(2,4-dinitrophenyl), And several other diphenyl oxalates, p-Chloromagnessium bromide (Grignard reaction), trans-1-(2′-methoxyvinyl)pyrene, Diphenoyl Peroxide, Adamantylidene Adamantane 1,2-dioxetan, Dimethyl indole hydroperoxide, rubrene, diphenylanthracenes, phenylethylanthracenes

4. Natural and synthetic fluorescent & laser dyes.

5. Thin layer Chromatography (TLC) self-visualization system, consist of peroxide, fluorescent dye (present in the silica mixture) and precursor for chemiluminescence such as oxalates.
